(19) United States
(12) Patent Application Publication
(10) Pub. No.: US 2007/0104762 A1
(43) Pub. Date: May 10, 2007

(54) Appetizer supplement to suppress age-related decline in capacity and appetite

(76) Inventor: Michael F. Roizen, Shaker Heights, OH (US)

Correspondence Address: ROBERTS, MARDULA & WERTHEIM, LLC, 11800 SUNRISE VALLEY DRIVE, SUITE 1000, RESTON, VA, 20191, US

(21) Appl. No.: 11/592,050

(22) Filed: Nov. 2, 2006

Related U.S. Application Data
(60) Provisional application No. 60/733,394, filed on Nov. 4, 2005.

(51) Int. Cl. A61K 47/00; A23G 3/00

(52) U.S. Cl. 424/439; 426/660

(57) ABSTRACT

A method for administering daily micronutrient requirements (DMRs) comprising vitamins, minerals and essential fatty acids targeted to reduce appetite and/or the risks associated with aging. The DMRs are administered to individuals via food products. In an embodiment of the present invention, the DMRs are offered in the form of a health bar. The DMRs may be incorporated into other forms without departing from the scope of the present invention. By way of illustration and not as a limitation, the constituents may be incorporated into a fruit or yogurt “smoothie,” a frozen desert such as an ice cream or sorbet, and beverage.

### CROSS REFERENCE TO RELATED APPLICATION

This application claims priority benefit under 35 U.S.C. §119(e) from provisional application No. 60/733,394, filed Nov. 4, 2005, which is incorporated by reference herein in its entirety for all purposes

### FIELD OF THE INVENTION

This application relates generally to nutritional supplements.

### BACKGROUND INFORMATION

Aging affects all parts of the human system. Bones can become thinner and less dense, joints may fail because of loss of cartilage, the circulatory system may become clogged, the heart may lose muscle or strength of contraction (or even fail), and mental-processing speed or capacity may decline. However, not all individuals with the same chronological age reflect the same “physiological” age. Thus, while aging cannot be prevented, the risks that arise from aging are not pre-ordained.

It has been shown that a healthy diet, regular physical activity program and positive attitude can help delay the onset and slow the progression of many age-related changes. What constitutes a “healthy” diet is an often debated topic and the subject of books and talk-shows. What is generally accepted is that it is not always easy for individuals to maintain a healthy diet or to acquire the nutrients that are beneficial for good health.

To many individuals, convenience foods are an attractive alternative to purchasing fresh foods and preparing meals. However, convenience foods are often high in calories and low in nutrients and fiber. Nutritional supplements in pill or tablet form may be helpful but must be taken regularly and provide little if any culinary gratification.

What would be desirable is an appetizer nutritional supplement (ANS) comprising specific portions of daily micronutrient requirements (e.g., vitamins, minerals, essentially fatty acids) targeted to reduce the risks associated with aging that is consumable in a form that is both tasty and palatable, as for example, before a meal. Such an ANS may also provide appetite suppressant effects.

### SUMMARY OF THE INVENTION

One embodiment provides a method for administering daily micronutrient requirements (DMRs) made up of vitamins, minerals and essential fatty acids targeted to reduce risks associated with aging, and/or decrease the speed of aging. The DMRs are administered to individuals via food products. According to one embodiment, the DMRs are offered in the form of a health bar. In general, the DMRs may be embodied as comestibles having other form factors without departing from the scope of the invention. By way of illustration and not as a limitation, the constituents may be incorporated into a “smoothie” (fruit, vegetable, nut oil, or yogurt based), a frozen desert (e.g., ice cream or sorbet), and a beverage.

In an exemplary embodiment, the DMRs are combined with other ingredients to form an appetizer nutritional supplement (ANS) that is consumed before a meal. Used in this way, the ANS also serves to suppress appetite by both specific means (releasing cholycystokinin and/or stimulating vagal afferents or as yet undetermined mechanisms. The DMRs embodied as an ANS may be varied in composition to suit the needs of particular groups, for example but not as a limitation, statin users, non-statin users, men and women.

Thus, one mode of practice of the invention is to provide an appetizer nutritional supplement (ANS) containing daily micronutrient requirements that include vitamins, minerals and essentially fatty acids targeted to reduce the risks associated with aging, and which is consumable in a form that is pleasing in flavor, texture, and consistency, as for example, before a meal.

Another embodiment provides an appetizer nutritional supplement that suppresses appetite.

Another embodiment provides an appetizer nutritional supplement that suppresses appetite via release of cholycystokinin and/or stimulating vagal or other nervous afferents or efferents

Another embodiment tailors the content of an appetizer nutritional supplement to the needs of particular groups of persons.

Another embodiment provides an appetizer nutritional supplement in the form of a health bar.

Another embodiment provides an appetizer nutritional supplement in the form of a frozen desert.

Another embodiment provides an appetizer nutritional supplement in the form of a beverage.

It is even another mode of practice of the invention to provide an appetizer nutritional supplement in the form of a “smoothie” type product.

A supplement according to at least some embodiments may be directed to reducing the risk of age related diseases such as cancer and arterial aging diseases including heart disease, sudden death from arrhythmias, stroke, memory loss, impotence and even wrinkled skin, and/or age related decline in functional capacity in cardiovascular or nervous or immune or musculosketal systems.

These modes of practice, as well as the various embodiments discussed will become apparent from a review of the detailed and general descriptions of the present application.

### DETAILED DESCRIPTION

An embodiment of the present invention provides a method for administering daily micronutrient requirements (DMRs) composed of vitamins, minerals and essential fatty acids targeted to reduce the risks associated with aging. The DMRs are administered to individuals via food products. In one embodiment, the DMRs are offered in the form of a health bar. The DMRs may be incorporated into other forms without departing from the scope of the invention. By way of illustration and not as a limitation, the DMRs may be incorporated into a fruit or yogurt “smoothie,” a frozen desert such as an ice cream or sorbet, and a beverage.

According to another embodiment, the DMRs are combined with other ingredients to form an appetizer nutritional supplement (ANS) that is consumed before a meal. In this way, the ANS also serves to suppress appetite, by direct action or indirectly such as releasing cholycystokinin or stimulating the afferent vagas nerve by nutritional means or by other nervous or endocrine mechanisms.

### EXAMPLE 1

The DMRs to be put in an ANS may be varied to suit the needs of particular groups. In a first example embodiment, an ANS for a statin user in the form of a health bar comprises ½ ounce walnuts, three ground dark chocolate nubbins, ground no-calorie carmel, fillers that are high in fiber. The DMRs to be included in the example embodiment's health bar are vitamins as listed in Table 1, below. It is noted that folate is commonly referred to as “vitamin F” (alternately termed a B vitamin by some authorities) and is the anion form of folic acid.

TABLE 1
Vitamins for 1^{st} example embodiment.

| VITAMIN | APPROX. AMOUNT |
|---|---|
| A | up to 2500 IU |
| B₆ | 2 mg |
| B₁₂ | 400 mcg |
| C | 50 mg |
| D | up to 1000 IU |
| E | 50 IU |
| F (folate) | 400 mcg |
| Thiamin | 12.5 mg |
| Riboflavin | 12.5 mg |
| Niacin | 15 mg |
| Biotin | 150 mg |
| Pantothenic acid | 150 mg |

The DMRs for the first example embodiment's health bar also include minerals as listed in Table 2, below.

TABLE 2
Minerals for 1^{st} example embodiment.

| MINERALS | APPROX. AMOUNT |
|---|---|
| Calcium | up to 1600 mg |
| Magnesium | up to 400 mg |
| Selenium | 100 mcg |
| Zinc | 7.5 mg |
| Potassium | 0 mg |

The DMRs for the first example embodiment's health bar also include vitamin-like substances as listed in Table 3, below.

TABLE 3
Vitamin-like substances for 1^{st} example embodiment.

| VITAMIN-LIKE | |
|---|---|
| SUBSTANCES | APPROX. AMOUNT |
| Lycopene | 30 mg |
| Lutein | 20 mg |
| Alpha lipoic acid | up to 200 mg |
| Acetyl-L-carnitine | up to 1500 mg a day |

### EXAMPLE 2

According to a second example embodiment of the invention, an ANS for statin users further includes up to 1200 mg of Coenzyme Q10 as an ingredient, in addition to those listed above for the first example embodiment.

### EXAMPLE 3

According to a third example embodiment of the invention, the ANS includes the ingredients described in the first example, with each of the identified DMRs being in the range of 25-100% of the minimum daily requirements as established by the government (e.g., the U.S. Food and Drug Administration) for each such DMR.

### EXAMPLE 4

According to a fourth example embodiment of the invention, the ANS includes the ingredients described in the first example, with an alternative formulation being substituted for the ½ ounce of walnuts. Specifically, up to a gram of three fatty acids (alone or in combination) are substituted for actual walnuts. These three fatty acids are docosahexaenoic acid (DHA), eicosapentaenoic acid (EPA), and alpha-linolenic acid (ALA). ALA is found in walnuts, and DHA and EPA are related to ALA and have demonstrated cardiac health benefits.

### EXAMPLE 5

According to a fifth example embodiment of the invention, the ANS includes the ingredients described in the first example, except substituting blueberry extract for the carmel.

### EXAMPLE 6

According to a sixth example embodiment of the invention, the ANS includes the ingredients described in the first example, except substituting raspberry extract for the carmel.

### EXAMPLE 7

According to a seventh example embodiment of the invention, the ANS includes the ingredients described in the first example, with each of the identified DMRs being substantially the same, with the exception of vitamins C and E. For the seventh example, the quantities of vitamins C and E are boosted, as listed in Table 4, below.

TABLE 4
Vitamin quantities for 7^{th} example embodiment.

| VITAMIN | APPROX. AMOUNT |
|---|---|
| C | 500 mg |
| E | 500 IU |

### EXAMPLE 8

According to an eighth example embodiment of the invention, the ANS includes the ingredients described in the first example, with each of the identified DMRs being substantially the same and further including additional DMRs identified in the listing in Table 5, below.

TABLE 5
Vitamin-like substances for 8^{th} example embodiment.

| VITAMIN-LIKE | |
|---|---|
| SUBSTANCES | APPROX. AMOUNT |
| 1-arginine | up to 70 mg |
| tyrosine | up to 180 mg |
| taurine | up to 70 mg |
| green tea catechins | up to 120 mg |
| caffeine | up to 20 mg |
| querecin | up to 10 mg |

The green tea catechins as a class may be used as an ingredient in the ANS, as listed in this example. Of the green tea catechins, epigallocatechin gallate (EGCG) is currently identified as a particularly useful anti-oxidant.

It has been demonstrated that consuming walnuts prior to a meal, at the levels indicated for some above examples, inhibits stomach emptying and suppresses appetite, and this and other substances consumed can stimulate release of cholycystokinin and or vagal or other nerve impulses that inhibit stomach emptying and or hunger.

One embodiment provides a method of reducing appetite when an ANS, including as ingredients walnuts, ground dark chocolate nubbins, ground no-calorie carmel, fillers that are high in fiber, and DMRs, is eaten 8 to 30 minutes before a meal. The appetite suppression affect, particularly of the walnut content of the ANS, is effective to reduce the amount of food consumed during the meal. It is understood that the mechanism of action that brings about the reduction in appetite is induced release of endogenous hormones or gut polypeptides or other substances that reduce appetite.

Sustained use of the ANS according to the method of reducing appetite is effective to support weight loss and reduction of waist size.

Studies have indicated that the combination of vitamins and minerals and micronutrients decreases sudden death and delays arterial aging. Studies further indicate that the combination of vitamins and minerals and micronutrients additionally provides some degree of protection against certain cancers.

Accordingly, another facet of the invention is its use to reduce the risks and/or processes associated with aging, through administration of nutritional supplements in the form of an appetizer bar (or other delivery mechanism) containing daily micronutrient requirements that include targeted vitamins, minerals and essentially fatty acids. To reduce the risks associated with aging, an ANS according to the various embodiments supports heart function, supports arterial health, supports health of a subject's immune system, and supports reduction of defects in mitochondrial function.

The delivery mechanism is selected from among various diverse choices such that at least one mechanism will be pleasing in flavor, texture, and consistency to most any subject. Smoothies, frozen desserts, or beverages are all strategically available as delivery mechanisms for nutritional supplements delivered according to embodiments of the present invention.

Another embodiment provides a method for packaging a dietary supplement targeted to reducing the risks and or processes associated with aging. In this embodiment, an ANS comprises walnuts, ground dark chocolate nubbins, ground no-calorie carmel, fillers that are high in fiber, and DMRs in the form of a health bar. The health bar is packed in an airtight container. By way of illustration, and not as a limitation, the health bar is packed in an aluminized polyester film in the presence of nitrogen gas and dated for shelf life. Dry nitrogen gas (i.e., without water vapor) is particularly effective for practicing this embodiment. Although nitrogen is named as an example, other gasses that are relatively non-reactive are suitable. As a further illustration, and not as a limitation, the health bar is packed in the airtight container with a preserving substance added to the bar to preserve fats in the bar.

An appetizer bar to suppress age-related disease and appetite has been shown. It will be understood by those skilled in the art that the present invention may be embodied in other specific forms without departing from the scope of the invention disclosed and that the examples and embodiments described herein are in all respects illustrative and not restrictive. Those skilled in the art of the present invention will recognize that other embodiments using the concepts described herein are also possible. Further, any reference to claim elements in the singular, for example, using the articles “a,” “an,” or “the” is not to be construed as limiting the element to the singular.

What is claimed is:

1. A nutritional supplement product comprising:
a nutritional supplement bar comprising:
walnuts;
ground dark chocolate;
fiber rich fillers; and
a binder substance selected from the group consisting of:
ground no-calorie carmel, blueberry extract, and raspberry extract; and
a package sealingly enclosing the bar with a preserving substance so as to preserve the nutritional supplement bar.

2. The nutritional supplement product according to claim 1, wherein the nutritional supplement bar further comprises at least one quarter of a daily required level of each of vitamin A, vitamin B₆, vitamin B₁₂, vitamin C, vitamin D, vitamin E, folate, Thiamin, Riboflavin, Niacin, Biotin, Pantothenic acid, Calcium, Magnesium, Selenium, Zinc, Potassium, Lycopene, and Lutein.

3. The nutritional supplement product according to claim 2, wherein the nutritional supplement bar further comprises at least one quarter of a daily required level of each of Alpha lipoic acid, and Acetyl-L-carnitine.

4. The nutritional supplement product according to claim 2, wherein the nutritional supplement bar further comprises Coenzyme Q10.

5. The nutritional supplement product according to claim 2, wherein the nutritional supplement bar further comprises 1-arginine, tyrosine, taurine, green tea catechin, caffeine, and querecin.

6. The nutritional supplement product according to claim 1, wherein the preserving substance comprises a substantially non-reactive fluid.

7. The nutritional supplement product according to claim 6, wherein the substantially non-reactive fluid comprises nitrogen gas.

8. The nutritional supplement product according to claim 1, wherein the preserving substance is added to the nutritional supplement bar.

9. A nutritional supplement for a statin user, wherein the nutritional supplement is sweetened without adding sugar, and wherein the nutritional supplement comprises:
ground walnuts;
ground dark chocolate;
a binder substance selected from the group consisting of: ground no-calorie carmel, blueberry extract, and raspberry extract;
high fiber filler;
vitamin A;
vitamin B₆;
vitamin B₁₂;
vitamin C in a dosage sufficiently low so as to not interfere with the anti-inflammatory effect of statins;
vitamin D;
vitamin E in a dosage sufficiently low so as to not interfere with the anti-inflammatory effect of statins;
folate;
Thiamin;
Riboflavin;
Niacin;
Biotin;
Pantothenic acid;
Calcium;
Magnesium;
Selenium;
Zinc;
Lycopene; and
Lutein.

10. The nutritional supplement for a statin user according to claim 9, wherein the nutritional supplement is provided in a form selected from the group consisting of: a health bar, a beverage, a frozen desert, and a smoothie.

11. The nutritional supplement for a statin user according to claim 9, wherein the amount of each of the vitamin A, vitamin B₆, vitamin B₁₂, vitamin C, vitamin D, vitamin E, folate, Thiamin, Riboflavin, Niacin, Biotin, Pantothenic acid, Calcium, Magnesium, Selenium, Zinc, Lycopene, and Lutein, is at least one fourth of a daily requirement level.

12. The nutritional supplement for a statin user according to claim 11, wherein the nutritional supplement further comprises at least one fourth of a daily requirement level of Alpha lipoic acid and Acetyl-L-carnitine.

13. The nutritional supplement for a statin user according to claim 9, further comprising up to about 1200 mg of Coenzyme Q10.

14. The nutritional supplement for a statin user according to claim 9, wherein consumption of the nutritional supplement supports function of statins in reducing inflammation.

15. The nutritional supplement for a statin user according to claim 14, wherein consumption of the nutritional supplement further supports appetite suppression.

16. The nutritional supplement for a statin user according to claim 9, further comprising 1-arginine, tyrosine, taurine, green tea catechin, caffeine, and querecin.

17. A nutritional supplement for a statin user, the nutritional supplement comprising:
ground walnuts;
ground dark chocolate;
ground no-calorie carmel;
high fiber filler;
up to about 2500 IU of vitamin A;
about 2 mg of vitamin B₆;
about 400 mcg of vitamin B₁₂;
about 50 mg of vitamin C;
up to about 1000 IU of vitamin D;
about 50 IU of vitamin E;
about 400 mcg of folate;
about 12.5 mg of Thiamin;
about 12.5 mg of Riboflavin;
about 15 mg of Niacin;
about 150 mg of Biotin;
about 150 mg of Pantothenic acid;
up to about 1600 mg of Calcium;
up to about 400 mg of Magnesium;
about 100 mcg of Selenium;
about 7.5 mg of Zinc;
about 30 mg of Lycopene;
about 20 mg of Lutein;
up to about 200 mg of Alpha lipoic acid;
up to about 1500 mg of Acetyl-L-carnitine;
up to about 400 mg of Coenzyme Q10;
up to about 70 mg 1-arginine;
up to about 180 mg tyrosine;
up to about 70 mg taurine;
up to about 120 mg green tea catechin;
up to about 20 mg caffeine; and
up to about 10 mg querecin.

18. A nutritional supplement for a non-statin user, the nutritional supplement comprising:
ground walnuts;
ground-dark chocolate;
ground no-calorie carmel;
high fiber filler;
up to about 2500 IU of vitamin A;
about 2 mg of vitamin B₆;
about 400 mcg of vitamin B₁₂;
about 500 mg of vitamin C;
up to about 1000 IU of vitamin D;
about 500 IU of vitamin E;
about 400 mcg of folate;
about 12.5 mg of Thiamin;
about 12.5 mg of Riboflavin;
about 15 mg of Niacin;
about 150 mg of Biotin;
about 150 mg of Pantothenic acid;
up to about 1600 mg of Calcium;
up to about 400 mg of Magnesium;
about 100 mcg of Selenium;
about 7.5 mg of Zinc;
about 30 mg of Lycopene;
about 20 mg of Lutein;
up to about 200 mg of Alpha lipoic acid;
up to about 1500 mg of Acetyl-L-carnitine;
up to about 400 mg of Coenzyme Q10;
up to about 70 mg 1-arginine;
up to about 180 mg tyrosine;
up to about 70 mg taurine;
up to about 120 mg green tea catechin;
up to about 20 mg caffeine; and
up to about 10 mg querecin.

19. A method for promoting health and suppressing appetite comprising:
consuming a nutritional supplement comprising:
walnuts,
ground dark chocolate,
ground no-calorie or low-calorie carmel or cinnamon, and
fiber rich fillers; and
consuming a meal about 8 to 30 minutes subsequent to consuming the nutritional supplement.

20. The method of claim 19, wherein consistent repetition of the method by a subject supports heart function.

21. The method of claim 19, wherein consistent repetition of the method by a subject supports arterial health.

22. The method of claim 19, wherein consistent repetition of the method by a subject supports immune system health.

23. The method of claim 19, wherein consistent repetition of the method by a subject supports weight loss.

24. The method of claim 19, wherein consistent repetition of the method by a subject supports reduction of waist size.

25. The method of claim 19, wherein consistent repetition of the method by a subject reduces defects in mitochondrial function.

26. The method of claim 19, wherein consistent repetition of the method by a subject supports function of statins in reducing inflammation.

27. The method of claim 19, wherein appetite is suppressed by releasing cholcystokinine or stimulating the vagus nerve or other nerves.

28. A method for suppressing appetite comprising:
consuming a nutritional supplement comprising walnuts; and
consuming a meal about 8 to 30 minutes subsequent to consuming the nutritional supplement, such that appetite is inhibited.

29. The method of claim 28, wherein appetite is inhibited by releasing cholcystokinine or stimulating the vagus nerve or other nerves.
